(19) United States
(12) Patent Application Publication
(10) Pub. No.: US 2007/0129677 A1
(43) Pub. Date: Jun. 7, 2007

(54) Double V-shaped groove safety syringe

(76) Inventor: Hsiu-Yun Lin, Pingzhen City (TW)

Correspondence Address: RABIN & BERDO, P.C., Suite 500, 1101 14 Street, N.W., Washington, DC, 20005, US

(21) Appl. No.: 11/295,655

(22) Filed: Dec. 7, 2005

(51) Int. Cl. A61M 5/00

(52) U.S. Cl. 604/110; 604/197

(57) ABSTRACT

A “double V-shaped groove safety syringe” is a needle protective mechanism for casing a commonly used syringe, comprising of an outer needle barrel and an adapter. The characteristic of the invention lies in which the outer needle barrel is a cylinder for casing a hollow fluid chamber and is able to move up or down. The outer needle barrel has an adapter set inside, where one end of the adapter receives an exhaust portion of a hollow fluid chamber and the other end of the adapter receives a needle portion. Said outer needle barrel and the adapter of the invention enables the adapter and the needle portion to be retained inside the outer needle barrel after said syringe structure is used for injection and the hollow fluid chamber is drawn out from the outer needle barrel, so as to prevent others from being infected through means of a second injection from a used needle.

### BACKGROUND OF THE INVENTION

I. Field of the Invention

The present invention refers to a “double V-shaped groove safety syringe”, particularly to a disposable safety needle structure tied with a commonly used syringe, to prevent others from being infected through means of a second injection from a used needle. The invention is composed of an outer needle barrel and an adapter, wherein the outer needle barrel cases a hollow fluid chamber and moves up or down in addition to disposal of said adapter. Moreover, one end of the adapter receives an exhaust portion of said hollow fluid chamber and the other end thereof receives a needle.

II. Description of the Prior Art

Generally, a syringe needle has to be covered by a good protective mechanism after each use, in order to prevent anyone from being infected by a second injection from a used needle. Moreover, those syringes that have no needle protective devices are no longer sold for use under stipulations and laws in many countries over the world. Therefore, a disposable safety needle structure has become a subject to be improved and solved.

In view of the foregoing drawbacks, a safety syringe structure with a double V-shaped groove of the invention is disclosed herein with more practicability in the field to improve a conventional problem of a disposable safety syringe structure.

The main objective of the present invention is to provide a safety syringe with a double V-shaped groove that has the advantages of a simplified structure and is convenient and safe to use. In addition, the invention is able to match up with a conventionally used syringe, so as to reduce the syringe manufacturing cost and expand the syringe applications at the same time.

Another objective of the invention is to provide a safety syringe with a double V-shaped groove; thereby a used needle is unable to be taken out without external force.

Another objective of the invention is to provide a safety syringe with a double V-shaped groove that enables a ring to be disposed outside a hollow portion on the front end of a conical ring of the adapter. The internal wall of the ring comprises of a double V-shaped groove for screwing a needle therein in the direction of either clockwise or anti-clockwise.

### SUMMARY OF THE INVENTION

To achieve the foregoing objectives, said “double V-shaped groove safety syringe” of the invention is a needle protective mechanism for receiving a syringe for use and is composed of an outer needle barrel and an adapter. The characteristic of the invention lies in the outer needle barrel acting as a cylinder for casing a hollow fluid chamber and is able to move up or down. The invention enables a set of forward clamps and a set of reverse clamps to be projectively disposed on the internal wall of the needle barrel on the rear end thereof opposed to one another. In addition, it has a set of retaining clamps disposed on the internal wall of the needle barrel on the front end thereof. The adapter is disposed inside of said outer needle barrel, with a conical ring shaping bigger on the front and smaller on the rear end thereof disposed on the middle of the adapter. The adapter has a hole on the rear end receiving an exhaust portion of the hollow fluid chamber and has a hollow portion disposed on the front end receiving a needle portion; the hole and the hollow portion are connected for fluid to put through.

Said syringe structure with a double V-shaped groove enables a ring to be disposed outside the hollow portion on the front end of said conical ring of the adapter and the internal wall of the ring comprises of a double V-shaped groove for screwing a needle portion in the direction of either clockwise or anti-clockwise.

Accordingly, after said syringe structure is used for injection and the hollow fluid chamber is drawn out from the outer needle barrel, the design of said outer needle barrel and the adapter of the invention enables the adapter and the needle portion on the front end of the adapter to be retained inside the outer needle barrel, so as to prevent others from being infected through means of second injection from a used needle.

### BRIEF DESCRIPTION OF THE DRAWINGS

FIG. 1 is an assembly view showing an embodiment of the invention.

FIG. 2 is an exploded view of the invention.

FIG. 2-A is a schematic view showing the double V-shaped groove inside the ring of the present invention.

FIG. 3 is a cross-sectional view showing the integration of present invention and a syringe.

FIG. 4 is a cross-sectional view showing the separation of present invention and a syringe.

### DESCRIPTION OF THE PREFERRED EMBODIMENT

Further aspects, objects, and desirable features of the invention will be better understood from the detailed description and drawings that follow in which various embodiments of the disclosed invention are illustrated by way of examples.

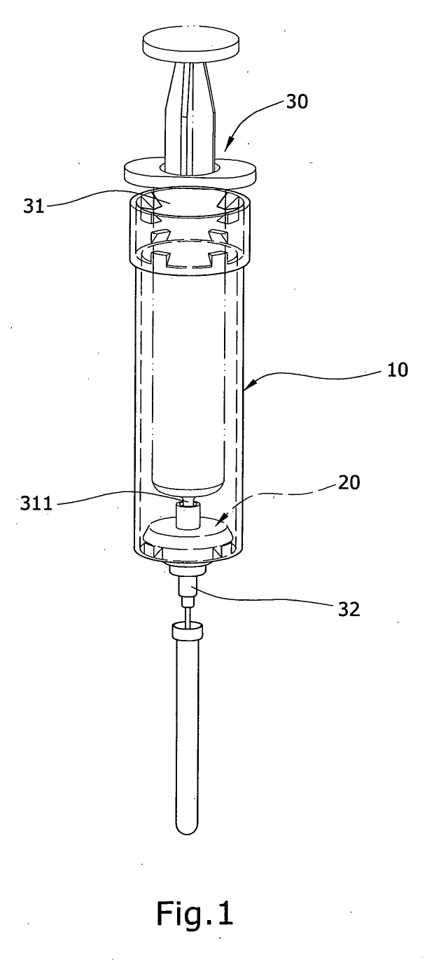

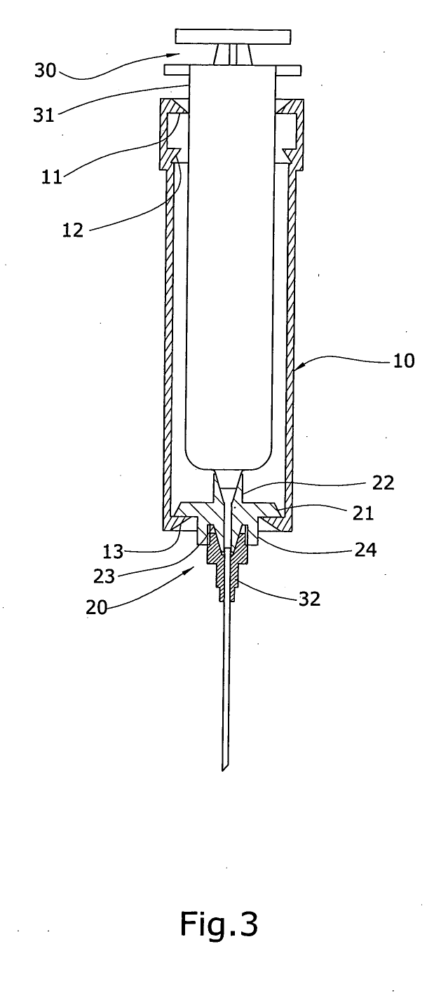

With reference to FIGS. 1, 2 and 3, said safety syringe with a double V-shaped groove of the invention is a needle protective mechanism receiving a commonly used syringe 30 for use, mainly comprising of an outer needle barrel 10 and an adapter 20. The characteristic of the invention lies in that the outer needle barrel 10 is a cylinder for casing a hollow fluid chamber 31 and is able to move up or down. The invention has a set 20 of forward clamps 11 and a set of reverse clamps 12 projectively disposed and opposed to one another on the internal wall of the needle barrel on the rear end. In addition, it has a set of retaining clamps 13 projectively disposed on the internal wall of the needle barrel on the front end thereof. The adapter 20 is disposed inside said outer needle barrel 10, with a conical ring 21 in the shape of forming bigger on the front. The smaller rear end of 25 the conical ring is disposed on the middle of the adapter 20 and the adapter 20 has a hole 22 on the rear end thereof receiving an exhaust portion 311 of said hollow fluid chamber 31 and has a hollow portion 23 disposed on the front end thereof receiving a needle portion 32; the hole 22 and the hollow portion 23 are connected for fluid to put through.

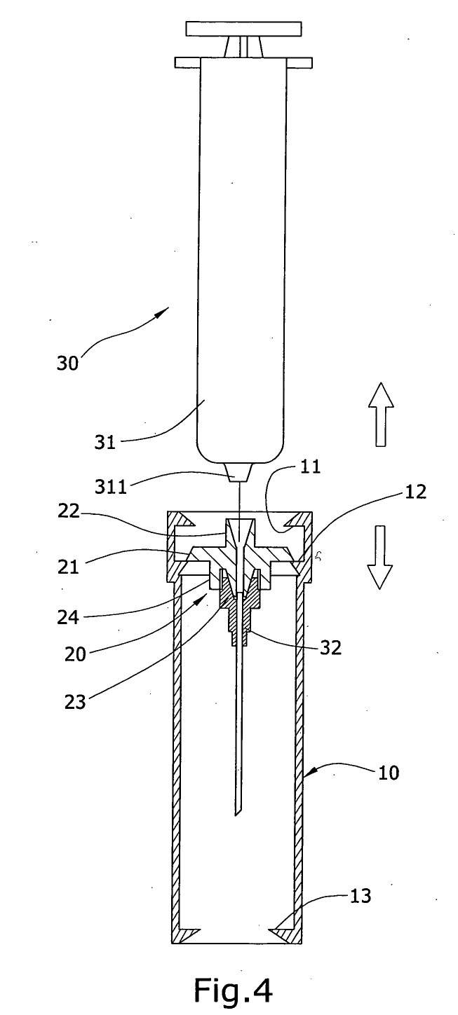

Accordingly, after the syringe 30 is used for injection and the hollow fluid chamber 31 is drawn out from the outer needle barrel 10 (as shown in FIG. 4), the adapter 20 and the needle portion 32 are both pulled backwards following said hollow fluid chamber 31. Consequently, when the adapter 20 is moved back to the rear end of the outer needle barrel 10, the conical ring 21 easily passes through the reverse clamps 12 and is stopped by the forward clamps 11, thereby enabling the adapter 20 and the needle portion 32 to be retained in-between the forward clamps 11 and the reverse clamps 12, i.e., the adapter 20 and the needle portion 32 on the front end of the adapter are retained inside the outer needle barrel 10, so as to prevent others from being infected through means of a second injection from the needle portion 32.

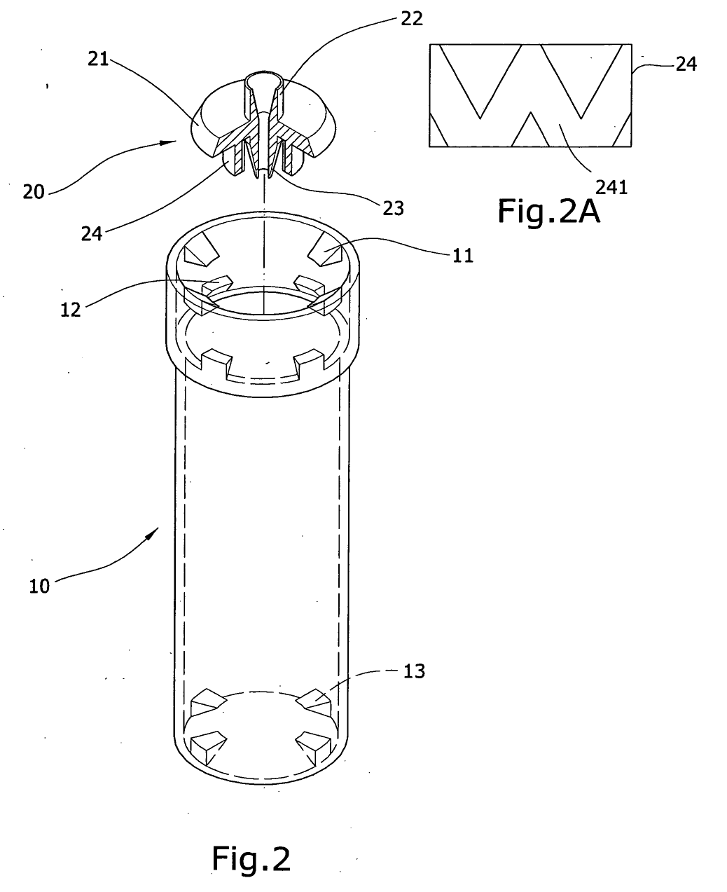

With reference to FIG. 2, a ring 24 is disposed outside the hollow portion 23 on the front end of said conical ring 21 of the adapter 20, the internal wall of the ring 24 comprises a double V-shaped groove 241, enabling the needle portion 32 to be easily received in the hollow portion 23 on the front end of the adapter 20. Moreover, the double V-shaped groove 241 enables said needle portion 32 to be easily screwed and fixed therein in the direction of either clockwise or anti-clockwise by a right-handed or a left-handed person for use.

In summary, the safety syringe with a double V-shaped groove of the invention indeed is able to store a used needle in the needle protective mechanism that is separated from said hollow fluid chamber, to prevent others from being infected through means of a second injection from a used needle.

New characteristics and advantages of the invention covered by this document have been set forth in the foregoing description. It is to be expressly understood, however, that the drawings are for the purpose of illustration only and are not intended as a definition of the limits of the invention. Changes in methods, shapes, structures or devices may be made in details without exceeding the scope of the invention by those who are skilled in the art. The scope of the invention is, of course, defined in the language in which the appended claims are expressed.

What is claimed is:

1. A safety syringe with a double V-shaped groove, comprising of a needle protective mechanism for casing a commonly used syringe for use, comprising of an outer needle barrel and an adapter, the characteristic lies in;
The outer needle barrel is a cylinder for casing a hollow fluid chamber and is able to move up or down; having a set of forward clamps and a set of reverse clamps projectively disposed and opposed to one another on the internal wall of the needle barrel on the rear end thereof and a set of retaining clamps projectively disposed on the internal wall of the needle barrel on the front end. The adapter is disposed inside said outer needle barrel, having a conical ring shaping bigger on the front and smaller on the rear end of the conical ring disposed on the middle of the adapter, and having a hole on the rear end thereof receiving an exhaust portion of said hollow fluid chamber and having a hollow portion disposed on the front end thereof receiving a needle portion; the hole and the hollow portion are connected for fluid to put through.

2. The safety syringe with a double V-shaped groove of claim 1, wherein the conical ring of the adapter has a ring disposed outside the hollow portion on the front end thereof; the ring comprises of a double V-shaped groove on the internal wall thereof.
